(19) United States
(12) Patent Application Publication
(10) Pub. No.: US 2021/0330988 A1
(43) Pub. Date: Oct. 28, 2021

(54) Portable Apparatus for Laser Therapy

(71) Applicant: Richard Albright, Franklin, TN (US)

(72) Inventor: Richard Albright, Franklin, TN (US)

(21) Appl. No.: 16/858,792

(22) Filed: Apr. 27, 2020

(51) Int. Cl. A61N 5/06

(52) U.S. Cl. CPC A61N 5/0616; A61N 2005/067

(57) ABSTRACT

A portable apparatus for laser therapy surgery comprising a readily portable housing, a plurality of laser sources of same or different wavelengths provided in the housing, and a flexible waveguide extending from the housing to transmit laser light from each of the plurality of laser sources to target area. A camera system to provide instructions remotely to the plurality of the users, a central processing unit communicating with a communication terminal to receive data regarding a purchase of one or more treatment credits by the user, the purchased treatment credits to the apparatus associated with the user. A computer system is configured to connect to the apparatus remotely via internet servers to receive plurality of data received from the user and apparatus, wherein the plurality of data comprising apparatus identification information associated with remotely located laser emitting apparatus and user identification information associated with a user of the apparatus.

### TECHNICAL FIELD

The present invention relates to a portable apparatus to perform laser therapy, and, more particularly, a portable device to provide plurality of laser sources of different wavelengths so that the device may provide different laser treatment to the patient depending upon the conditions of the patient.

### BACKGROUND

Laser light therapy has become increasingly popular in physiotherapy and surgery applications due to the many benefits available through the application of laser light. Laser light can be used to treat a variety of problems, ranging from relatively mild conditions, such as acne and skin wrinkling, to more complex problems lying deep under the skin, including afflictions of both organs and bones. In many cases, the application of laser therapy may negate the need for conventional pharmaceutical and/or surgical procedures. Different powers, wavelengths, and frequencies are used to target the distinct tissue types associated with the different conditions being treated. With the many physical benefits available over the large range of these powers, wavelengths, and frequencies, there exists a need for a device to deliver a large number of different combinations of these values in order to treat a wide variety of conditions. Further, medical office and clinical conditions, the device should be readily portable, updatable, and relatively easy to use.

A relevant art U.S. Pat. No. 8,888,830 B2, discloses dermatological medical devices and methods comprising a distal end for positioning at a region proximal a target therapeutic region of tissue, an output port at the distal end, an energy source that generates optical energy, which is output from the output port to the target therapeutic region of tissue, and a microcontroller that processes replenishment data that controls an operation parameter of the device. The device is activated for performing a treatment operation in response to a receipt and processing of the replenishment data. However, the device is not flexible or mobile as it cannot be remotely handled or controlled by the trainers or specific personnel.

In another relevant art, US20110144410A1 a therapy device for delivering electromagnetic radiation is disclosed which includes a body, an energy source for emitting a desired wavelength of electromagnetic radiation and a material dispensing system for dispensing a desired material for use with the device. The dispensing system of the invention is adaptable to receive a removable container for containing said desired material and said dispensing system having a sensor for reading an identifier disposed on said removable container. Moreover, the therapy device of the invention also includes a control mechanism for controlling the energy source and the material dispensing system, said control mechanism being in communication with said sensor for receiving information concerning said removable container. However, the device is not flexible or mobile as it cannot be remotely handled or controlled by the trainers or specific personnel. Furthermore, during the application of the therapy there is a need for a user interface to selectively choose any kind of particular protocol or treatment for the user.

Another relevant art US20080147431 A1 discloses methods and apparatus for providing a treatment device on a pay-per-use basis. The method disclosed in the cited prior art discloses delivering of the treatment device to a retailer. The treatment device of the invention requires at least one digital key from a plurality of digital keys to be operated. Moreover, the treatment device of the invention is being programmed to modify data on the at least one digital key to indicate use of the treatment device. The method further comprises delivering a first digital key from the plurality of digital keys to the retailer on a first date whereas the first digital key stores data indicative of a first duration of authorized use for the treatment device. The method discloses further delivering a second digital key from the plurality of digital keys to the retailer on a second date whereas the second digital key stores data indicative of a second duration of authorized use for the treatment device. However the device is not flexible or mobile as it cannot be remotely handled or controlled by the trainers or specific personnel. Furthermore, during the application of the therapy there is a need for a user interface to selectively choose any kind of particular protocol or treatment for the user.

In view of above aforementioned problems, a novel device is strictly needed which can be readily adaptable in home and medical offices to perform therapeutic treatment and surgery to a patient.

### SUMMARY OF THE INVENTION

The invention discloses a portable laser apparatus which is capable to provide laser treatment to the patient based on the condition of the patient. The apparatus may include plurality of laser sources and may be controlled to provide plurality of laser emissions depending upon therapeutic and surgical procedures. The apparatus may be provided with the camera system to provide live training to the doctors remotely with the internet servers.

In one of the preferred embodiment of the invention, the portable apparatus for laser therapy is disclosed. The apparatus may include a readily portable housing which is provided to enclose a plurality of laser sources of same or different wavelength, a flexible waveguide extending from the housing to transmit laser light from each of the plurality of laser sources to target area. The apparatus may be provided with the camera system to provide plurality of instructions to the users remotely. Further the apparatus may include a central processing unit which communicates with the communication terminal to receive data regarding purchase of one or more treatments by the user. The purchased treatment credit is used to authorize the apparatus to perform treatments corresponding to the purchase treatment credits. The apparatus includes the user interface for displaying a plurality of data associated with purchased treatment credits. The displaying of the plurality of data associated with the purchased treatment credits through the user interface allows the user to choose from the plurality of purchase options. The displaying of the plurality of data associated with purchased treatment credits through the user interface allows the user to track the usage of the treatments of the apparatus.

Further the apparatus may include a computer system which is configured to connect to the apparatus remotely via the internet sources. The computer system may be configured to receive plurality of data associated with the users, apparatus. A performance tracker is provided to track the usage of the treatments.

Another preferred embodiment of the invention states a method of operating the portable laser apparatus. The method discloses receiving of the apparatus identification information associated with the located apparatus and user identification information associated with a user of the apparatus. Further, storing of the apparatus identification information and user identification information is done on the computer system via the internet. Once all the information is verified, the access to the apparatus is provided to the user so that the user is able to track the purchased treatment credits. The data associated with the purchased treatment credits authorizes the user to access other treatments based on the condition of the user. Moreover, the method also provides instructions to the users remotely through camera feature provided in the apparatus. The camera is configured to provide access to the users through internet.

### BRIEF DESCRIPTION OF THE DRAWINGS

The embodiments herein will be better understood from the following detailed description with reference to the drawings, in which:

FIG. 1 illustrates a perspective top sided view of portable laser emitting apparatus in accordance with an embodiment of the present invention.

FIG. 2 illustrates a bottom sided view of portable laser emitting apparatus in accordance with an embodiment of the present invention.

FIG. 3 illustrates an angular sectional or a left sided view of portable laser emitting apparatus in accordance with an embodiment of the present invention.

FIG. 4 illustrates an angular sectional or a right sided view of portable laser emitting apparatus in accordance with an embodiment of the present invention

FIG. 5 illustrates an isometric bottom sided view of the portable laser emitting apparatus displaying a camera system in accordance with an embodiment of the present invention.

FIG. 6 illustrates isometric view of the portable laser emitting apparatus in accordance with an embodiment of the present invention.

FIG. 7 illustrates a camera and a tablet system connected to the portable laser emitting apparatus.

### DETAILED DESCRIPTION OF THE INVENTION

The embodiments herein and the various features and advantageous details thereof are explained more fully with reference to the non-limiting embodiments that are illustrated in the following detailed description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The portable apparatus for laser therapy is disclosed in the claimed invention. Said apparatus is capable of providing laser light of different wavelengths depending upon the response of the patient. The apparatus may be provided with the housing to enclose plurality of laser sources which is capable of providing laser light of different wavelengths. The housing may include flexible waveguide which is extending from the housing to transmit laser light from each of the plurality of laser sources to target area. The apparatus is provided with the camera feature which is capable of providing instructions to the user remotely via the internet. The apparatus may include central processing unit which is configured to receive data related to purchase of treatment credits of the patient. The track of purchased treatment credits authorizes the apparatus to perform a number of treatments. The apparatus is actively coupled with a computer system to receive plurality of data associated with the apparatus and user identification. Moreover the data is also related to the track of usage of treatments. A performance tracker is provided to track the usage of treatments. The system is employed in medical and veterinary purposes depending upon the specific purpose of the treatment.

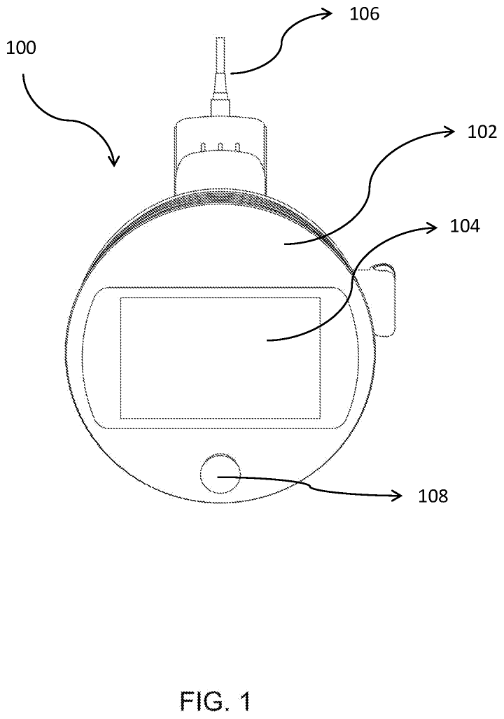

FIG. 1 illustrates a perspective top sided view of the portable apparatus (100) for laser therapy is disclosed in accordance with an embodiment. In top sided view of the apparatus a touch display (104) is installed on a top surface (102) to provide user interaction with the apparatus. A handle (108) is also displayed to move the apparatus (100) in any specified direction. A flexible waveguide (106) extending from the housing is shown and it is provided to emit laser light of different wavelengths. A hand piece with a battery changing system (110) is installed at the bottom side of the apparatus (100) and a combiner is attached to the hand piece with the battery changing system (110) for combining the laser lights of different wavelengths and then passed to the waveguide (106). The housing of the apparatus may contain a control switch (116) to activate a controller configured to run the apparatus according to the predetermined settings.

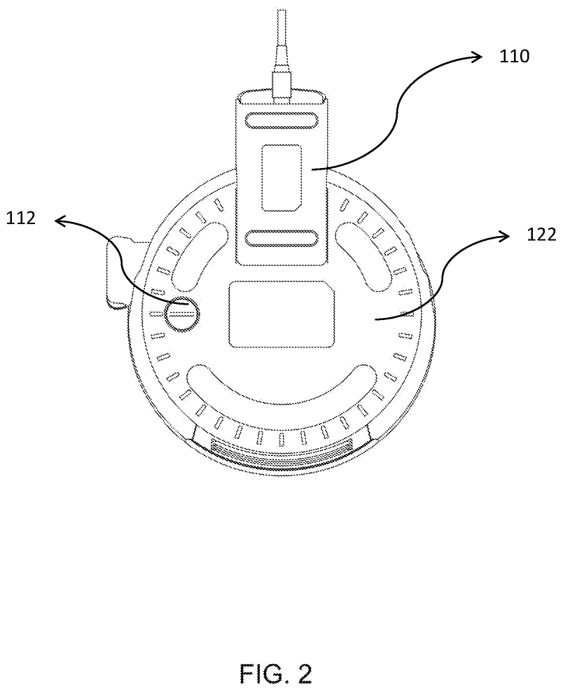

Referring to the FIG. 2, a bottom sided view of the apparatus is disclosed. The extended view of waveguide (106) is shown attached to the hand piece with the battery changing system (110). The waveguide (106) is shown to transmit laser light of different wavelengths. The combiner is provided to combine the laser lights from plurality of sources. An on/off or open/close switch is provided at a bottom plate (122) of the apparatus (122) to open or close the apparatus for the specified purpose.

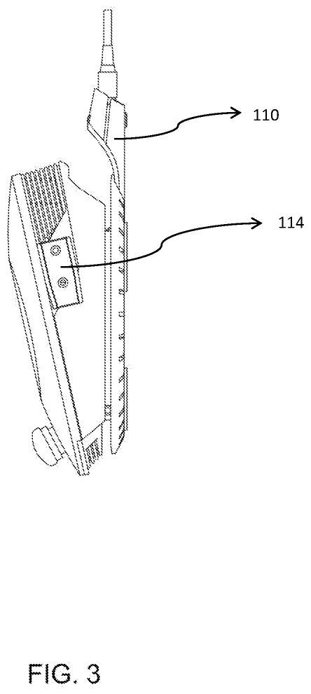

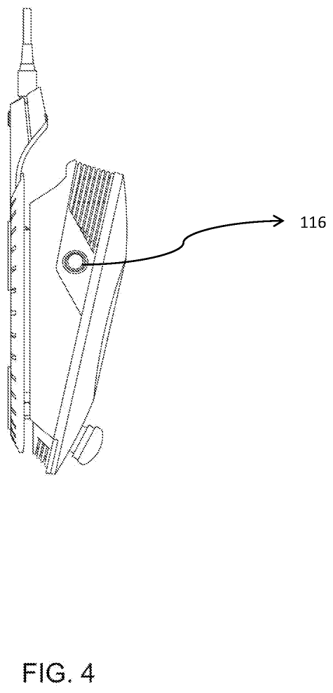

Referring to FIG. 3 and FIG. 4 an angular sectional or a left and right sided view of the apparatus (100) is illustrated. The waveguide is used to transmit laser light of different wavelengths. The handle (108) is also disclosed which is used to support the user so that the user display may be prevented from damage. The combiner is attached to the hand piece with the battery changing system (110) to combine the laser light of different lights. The hand piece with the battery changing system (110) is provided at the distal end of the waveguide. A communication terminal (114) is provided on the apparatus (100). The terminal (114) is configured to receive data updates for the controller and data storage. The communication terminal (114) may be, for example, a USB port used to connect the portable apparatus to a computer or other electronic device to download the update data. As another example, the communication terminal may be a wireless connector, such as a Wi-Fi connector to receive the update data in a wireless fashion. In FIG. 4 a control switch (116) is displayed. The control switch (116) is used to control the emission of the laser light from the apparatus (100) depending on the particular patient.

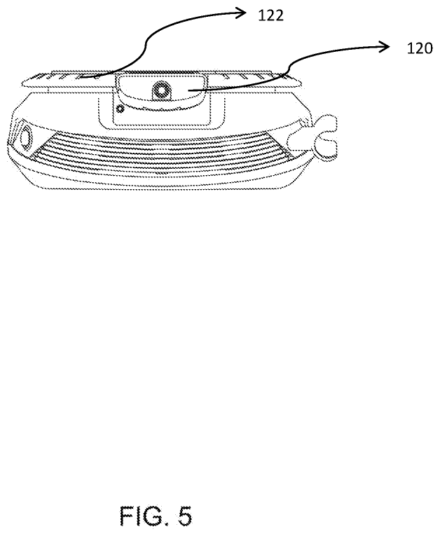

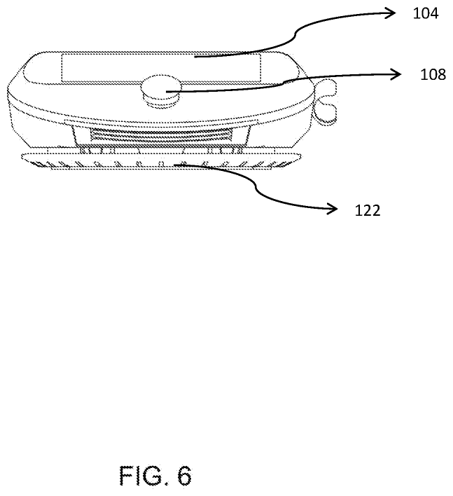

Referring to FIG. 5, an isometric bottom sided view of the apparatus is disclosed. The apparatus (100) is provided with a camera system (120) to view and train the trainees as required through an API or user interface. The camera system (120) enhances the treatment of the patients and provides plurality of instructions to the users remotely. Furthermore, instructions are provided to the users remotely through camera feature. The portable laser emitting apparatus (100) is provided with a power connection, such as an electrical cord, to be connected with an AC power source, as well as a rechargeable battery which may be provided inside or outside of the housing. The example hand piece with the battery changing system illustrated in FIG. 5 is provided with at least one switch (116) as shown in FIG. 4, to enable a user to switch between various laser application modes, and/or to start and stop emission of laser light from the hand piece with the battery changing system. In various example embodiments, the rechargeable battery may be removable from the portable laser emitting apparatus to be recharged, and in other various example embodiments the rechargeable battery may be recharged solely by power supplied by the AC power source. In other various exemplary embodiments of the present invention, the portable laser emitting apparatus may be equipped to receive power through wireless power transmission. Although a touch screen display (104) is illustrated in FIG. 6 which illustrates an isometric view of the apparatus (100), it will be understood by one skilled in the art that various other user interfaces may be employed in the portable laser emitting apparatus. For example, a conventional display and keypad combination may be substituted for the touch screen display. In various embodiments, the touch screen display can display images in 16 million color combinations.

The FIG. 6 illustrates the isometric view of the portable laser emitting apparatus (100). The flexible waveguide may be an optical cable, such as a single emitting fiber with a core size of approximately 600 um, and an NA of approximately 0.15 to 0.37. However, the waveguide is not limited to an optical cable, or an optical cable with these example attributes. The flexible waveguide may be provided with a pliable metal sheath surrounding the flexible waveguide to protect the flexible waveguide, and to house control wiring connected to the hand piece with the battery changing system.

In a preferred embodiment, a method for operating portable apparatus of providing laser therapy is disclosed. The pluralities of laser sources are provided to the apparatus for providing laser treatment of different wavelengths depending upon the condition of the patient. A patient specific protocol based on patient's condition is implemented in the system of operating the apparatus to provide desired treatment depending upon the condition of the patient. Moreover, the apparatus is configured in the manner to provide different laser treatments depending upon the responding of the patient. The system is developed in the manner to achieve the set of patient specific protocol that is capable to change the treatment of the patient according to the response of the patient. According to the embodiment of the apparatus illustrated in FIG. 1, a first laser source, having a wavelength of 660 nm, is provided to the optical coupler to serve as a guiding beam and/or a therapeutic laser source having stimulatory effects in superficial dermatological conditions such as open wounds, diabetic ulcers and infections. A plurality of laser sources are provided to the laser emitting apparatus, the outputs of which are combined by the combiner before the laser outputs enter the flexible waveguide and go on to pass through the coupler.

A second laser source may have a wavelength of 810 nm, with a power output ranging from 0.1 to 9 Watts, being in the NIR range, and centered at the peak of cytochrome c oxidase's absorption. The third laser source may have a wavelength of 980 nm, with a power output ranging from 0.1 to 12 Watts, also being in the NIR range, and centered at the peak of water's absorption. This third laser source may create thermal gradients on the cellular level along which blood would more readily flow. The fourth laser source may have a wavelength of 915 nm, with a power output ranging from 0.1 to 9 Watts, sitting at the peak of oxy-hemoglobin's absorption, thereby increasing the flow of oxygen from the blood to the cells for processing. It should be apparent to one skilled in the art that these are only some of the examples of useful laser wavelengths, and various other embodiments of the present general inventive concept may have any number of other combinations of laser sources, wavelengths, and/or powers. It is to be understood, however, that even though numerous characteristics and advantages of the present invention have been set forth in the foregoing description, together with details of the structure and function of the invention, the disclosure is illustrative only. Changes may be made in the details, especially in matters of shape, size, and arrangement of parts within the principles of the invention to the full extent indicated by the broad general meaning of the terms mentioned.

Some embodiments of the present invention may utilize pay per session/pay-per treatment model so that system of the claimed invention is capable to track the usage of treatments. The system is capable to limit the treatment time or usage of the apparatus, which must receive data regarding a purchase of one or more treatment credits by the user, wherein the purchased treatment credits authorize the apparatus to perform a number of treatments corresponding to the purchased treatment credits. According to various embodiments of the present invention, a number of analysis/or control operations such as, for example, treatment tracking/performance tracker, revenue estimation, credit-based activation may be available to the user of the apparatus remotely by the way of implementing plurality of servers. The pay per session or pay per treatment model is achieved by the way of above explained features. The remote servers are configured to track the usage of treatments remotely. It is understood that the plurality of servers make the apparatus an automated management process performed by a processor and memory configuration that is able to communicate data to and from the apparatus through for example, an internet or cellular connection. In various embodiments, pay per session model is achieved by implementing credit based system wherein the user purchases, through the GUI, a certain number of procedures or time of operation of the device from a remote control location. In various embodiments, the GUI may be displayed on the touch screen display or may be accessed on any other processing device having a display such as computer or smartphone. When displayed on a device other than the apparatus, the GUI may be controlled by the CPU of the apparatus through the communication terminal. In other words, various displays herein described may be displayed on, for example, a laptop computer that is in communicating remotely. In one of the preferred embodiment of the present invention, a purchase option display is disclosed. It may allow a user to choose from the plurality of purchase options. In some embodiments, the user may be provided with an entry window to enter a different quantity than those shown in predetermined packages.

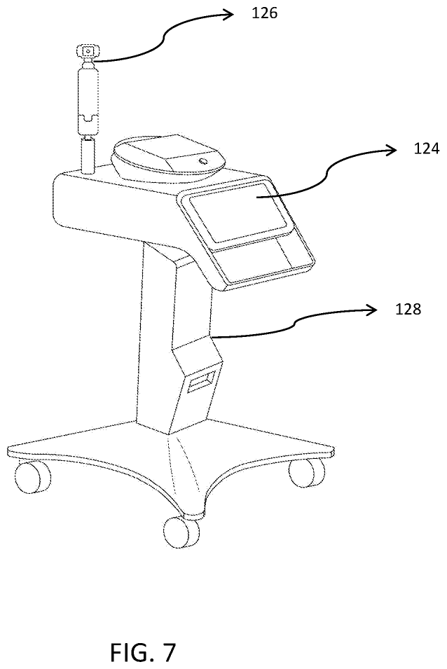

FIG. 7 illustrates a camera system (120/126) and a tablet (124) being attached to the apparatus (100) which is configured to view and train the trainee remotely. The whole system is supported on a stand (128). The camera (126) is configured to connect to the plurality of other devices remotely through the application program interface (API). A training module is configured to be remotely controlled by the trainers. The training module is configured to provide instructions to the plurality of users remotely through an application program interface. The application program interface (API) is configured to connect the plurality of servers to the apparatus. The APIs may include XML web service APIs for integration of system for providing live training to the doctors and for including camera feature and training module. All functionality available in the CSR Web application is provided with these API sets. The Java and XML-based APIs of the system may be configured to provide live training to the doctors remotely. The system can provide a customized interface to the system, or alternatively can provide laser therapy system developers with APIs and support in the integration effort.

The tablet (124) comprises a laser specific QR code to provide a number of instructions and modules of training and information to the user/trainer. The Laser specific QR code of the tablet (124) is configured to provide: schedule system training which includes providing a calendar schedule to a number of trainers; and schedule clinical training to provide clinical training to the trainers. Through the laser specific QR code the user can contact sales representative through a phone call or email to schedule a call depending upon the representative's calendar schedule. The user can contact summus directly via phone or email to schedule a specific call. The laser specific QR code also provides a contact technical support to provide the user with a copy of invoice, days remaining in warranty, schedule a support session through technical support calendar schedule, immediate summus support. Furthermore, a video chat is also provided for any specific practice. A number of training videos can also be provided through the QR code such as system training videos and clinical training videos. The training videos or training sessions are recorded into the tablet (124) through the specific laser QR code for record so that it can be browsed from the list of recorded training sessions when required. The whole information or scheduling and training sessions are provided through Summus Portal provided through the Laser Specific QR code of the tablet (124).

While the present invention has been illustrated by description of several example embodiments, it is not the intention of the applicant to restrict or in any way limit the scope of the inventive concept to such descriptions and illustrations. Instead, the descriptions, drawings, and claims herein are to be regarded as illustrative in nature, and not as restrictive, and additional embodiments will readily appear to those skilled in the art upon reading the above description and drawings

What is claimed is:

1. A portable apparatus for laser therapy surgery, the apparatus comprising:
a readily portable housing;
a plurality of laser sources of same or different wavelengths provided in the housing; and
at least one flexible waveguide extending from the housing to transmit laser light from each of the plurality of laser sources to target area;
a camera system to provide instructions remotely to a plurality of the users;
a central processing unit communicating with a communication terminal to receive data regarding a purchase of one or more treatment credits by the user, the purchased treatment credits to the apparatus associated with the user, wherein the purchased treatment credits authorize the apparatus to perform a number of treatments corresponding to the purchased treatment credits;
a performance tracker to track a number of usage of said treatments; and
a computer system configured to connect to the apparatus remotely via internet servers to receive plurality of data received from the user and apparatus, wherein the plurality of data comprising apparatus identification information associated with remotely located laser emitting apparatus, user identification information associated with a user of the laser emitting apparatus, inputs regarding the camera system.

2. The apparatus of claim 1, wherein the plurality of laser sources is configured to emit the plurality of laser light of desired wavelength on the target area according to the condition of the user.

3. The apparatus of claim 1, wherein a training module is configured to provide instructions to the plurality of users through trainers remotely through an application program interface, wherein the application program interface is configured to connect the plurality of servers to the apparatus.

4. The apparatus of claim 1, wherein the plurality of laser sources is configured to change the laser light of desired wavelength on the target area depending on the response of the patient.

5. The apparatus of claim 1, further comprising:
a hand piece with a battery changing system, provided at a distal end of the flexible waveguide, configured to emit the laser light to the target area;
a combiner to combine light from the plurality of laser sources into the laser light transmitted by the flexible waveguide; and
a carrying handle attached to the housing to provide support of carrying the device, to the user.

6. The apparatus of claim 1, further comprising:
a data storage configured to store a plurality of predetermined settings of wavelength and power combinations to be emitted from the plurality of laser sources, data corresponding to past usage of the apparatus, data associated with treatment credit information to validate user for treatment, user identification data; and
a controller configured to control the one or more laser sources to operate according to the predetermined settings in response to the apparatus being authorized to perform a treatment, the past usage data, to share the usage data and/or receive new treatment credits.

7. The apparatus of claim 1, wherein the communication terminal is configured to receive data updates for the controller and data storage.

8. The apparatus of claim 1, further comprising:
a user interface for displaying data associated with wavelength and power combinations, data corresponding to past usage of the apparatus, data associated with treatment credit information, user identification data;
a rechargeable battery to supply power to the apparatus; and
a switch, wherein the button is configured to operate the controller according to the predetermined settings.

9. The apparatus of claim 1, further comprising a touch screen display to provide user interaction with the apparatus.

10. A method of operating an apparatus for laser therapy surgery, the method comprising:
receiving, by the computer system, apparatus identification information associated with the remotely located apparatus and user identification information associated with a user of the apparatus;
storing, by the computer system, the apparatus identification information and user identification information;
communicating, by the computer system, with the apparatus associated with the user in response with the data associated the purchased treatment credits;
wherein the purchased treatment credits authorize the apparatus to perform treatments corresponding to the purchase treatment credits;
displaying, by the computer system, through a user interface, a plurality of data associated with purchased treatment credits; and
providing instructions, by the computer system to the plurality of users associated with the apparatus remotely through camera system.

11. The method of claim 10, wherein the receiving of data associated with user identification, purchased treatment credits, apparatus identification data is done through internet.

12. The method of claim 10, wherein the displaying of the plurality of data associated with the purchased treatment credits through the user interface allows the user to choose from the plurality of purchase options.

13. The method of claim 10, wherein the displaying of the plurality of the data associated with the purchased treatment credits through the user interface allows the user to track the usage of the treatments of the apparatus.

14. The method of claim 10, further comprising controlling, by the computer system, a user interface so as to be displayed on a remote device, wherein the data regarding the purchase of the one or more treatment credits by the user is facilitated through the user interface.

15. The method of claim 10, wherein the user interface is a web page displayed on an information processing device.

16. The method of claim 10, further comprising, prompting by the computer system, the user for the user identification information at an initial display of the user interface.

17. The method of claim 10, further comprising, displaying by the computer system, in the user interface a total number of treatments for which the laser emitting apparatus is currently authorized.

18. The method of claim 10, further comprising, displaying by the computer system, in the user interface an expiration date by which one or more of the corresponding authorized treatments must be performed.

19. The method of claim 10, further comprising, displaying by the computer system, in the user interface an option for the user to purchase additional treatment credits.
